(19) United States
(12) Patent Application Publication
(10) Pub. No.: US 2006/0200006 A1
(43) Pub. Date: Sep. 7, 2006

(54) METHOD AND APPARATUS FOR CONDUCTING AN ORAL EXAMINATION ON YOUTHFUL PATIENTS

(76) Inventors: Catherine Drogin, Brooklyn, NY (US); Peter Drogin, Brooklyn, NY (US)

Correspondence Address: MILBANK, TWEED, HADLEY & MCCLOY, 1 CHASE MANHATTAN PLAZA, NEW YORK, NY, 10005-1413, US

(21) Appl. No.: 11/383,974

(22) Filed: May 18, 2006

Related U.S. Application Data
(63) Continuation-in-part of application No. 10/617,935, filed on Jul. 11, 2003.

(51) Int. Cl. A61B 1/32

(52) U.S. Cl. 600/240

(57) ABSTRACT

This invention relates to a method for motivating cooperation of youthful patients during an oral examination and to an apparatus with which this can be achieved. The apparatus is an improved tongue depressor that bears graphical images printed directly onto, and covering the entirety of, one or more surfaces using non-toxic inks. The improved tongue depressor comprises a thin, flat non-edible stick with a longitudinal axis made of a non-toxic material such as wood. A doctor can use the improved tongue depressor to ease the fears of young patients by allowing patients to select favorite images. The patient's choice of image gives the doctor information from which the doctor can engage the patient in conversation, and to thereby redirect the patient's attention while completing an oral examination. By providing the patient with the improved tongue depressor of his or her choosing, the patient is further motivated to cooperate during the examination.

### RELATED CASES

This application is a continuation in part of U.S. patent application Ser. No. 10/617,935 filed Jul. 11, 2003, the disclosure of which is incorporated herein by reference.

### FIELD OF THE INVENTION

This invention relates to a method and apparatus for conducting a physical examination on youthful patients. More particularly, this invention relates to a method of motivating cooperation of youthful patients during an oral examination and to an improved tongue depressor with which this can be achieved.

### BACKGROUND OF THE INVENTION

Along with injections and dental drilling, oral examinations are vociferously feared and dreaded by many pediatric patients. The oral examination—a visual medical inspection of the oral cavity—is an integral part of a physical examination, which is performed on virtually every pediatric patient whether ill or well. Anxiety and fear generated by the anticipation and execution of the oral exam can create not only emotional distress for young patients and their parents but also places severe strain on the doctor-patient relationship. In addition, pediatric patients' fear-inspired resistance to the oral exam delays the process, thereby further hampering an already time-constrained doctors' productivity.

Even before hearing the request to “open your mouth and say aah,” the sight of a tongue depressor in a doctor's hand can send a child into a hysterical fit, prompting a desperate fight to prevent an oral examination from proceeding. This reaction is inspired by the patient's recollection from previous examinations of unpleasantness associated with the tongue depressor—most commonly an involuntary gagging reflex upon insertion of the depressor into the oral cavity and, more generally, the doctor's unwelcome invasion into a sensitive body part that occurs directly before the patient's eyes.

Consequently, pediatric practitioners must devise a method to gain patients' cooperation. These typically range from the promise of a reward (e.g., a sticker or lollipop) to physical restraint (e.g. a papoose board or parent intervention). The prospect of a subsequent reward, however, is often forgotten by younger patients in the presence of immediate discomfort. This is especially true if the reward cannot be enjoyed at the time of the exam, or if it is not constantly within the patient's view. The use of physical restraint is likely to amplify a patient's fear and discomfort at the time of the exam and creates a negative memory for future doctor's visits. These methods do little to quell patients' fear of the oral examination or the tongue depressor itself, thus perpetuating a fundamental strain in the ongoing relationship shared among patients, parents and doctors. As a result, the oral exam remains a traumatic experience, with the doctor seen as an inflictor of pain and the tongue depressor as an item to be feared.

Three primary categories of prior art tongue depressors arguably exist: those that incorporate candy or another child-friendly inducement as an integrated component or attachment; those treated with flavorings or other edible coatings; and those impregnated with a colorant. None, however, allow for any image to be found over the entire surface (top and/or bottom) of the depressor; i.e., over an area of the depressor that can, and is, put into the patient's mouth. In addition, none are designed to deliver immediate gratification to the patient immediately before, during, and after the execution of oral examination.

One of the key flaws in prior art tongue depressors bearing candy coatings or candy attachments is the patient's increased salivation resulting from the presence of the candy, which hinders the doctor's visual field, diminishes the blade's “grip” on the patient's tongue, and may interfere with various medical procedures including the taking of throat cultures. In addition, parental objection to bribing their child with candy, especially by a medical doctor who is suppose to know of the “dangers” of candy to children, is also a factor making these candy coated depressors less effective. Further, in some instances the depressor blade is longer due to the attachment of a candied element to the end of the blade held by the doctor, causing a likelihood that the patient's gag reflex will be over stimulated due to the doctor's over insertion of the now longer blade into the patient's mouth.

In addition to these physiological problems, several psychological disadvantages exist as well. With candy-enhanced depressors, the patient is rewarded before the need for cooperation has ended. Moreover, the reward is beyond the patient's field of vision during the exam and hence not perceptible to typically sight-minded young patients. Finally, it is expected that the doctor will give the patient the blade with the remaining confection on it for consumption after the oral exam's completion, which may complicate further examination.

U.S. Pat. No. 5,634,885, entitled ‘Tongue Depressor with Lollipop Holder,” issued to Kiro on Jun. 3, 1997, and is directed to a tongue depressor having a lollipop holder slot at the end of the blade of the depressor not intended to be placed into the patient's mouth, and a lollipop extending from the slot. While the '885 patent also discloses imprinting information/pictures onto the blade of the depressor, as can be seen in the '885 patent, any such information/picture is located directly proximate to the lollipop holder slot and so by definition is not intended to enter into the patient's mouth during examination.

U.S. Pat. No. 5,891,019, entitled ‘Tongue Depressor for Children and Method,” issued to Young et al. on Apr. 6, 1999, and is similarly directed to a tongue depressor 25 bearing an image 32, However, as with the '885 information disclosure, image 32 of the '019 patent is also specifically intended to be located along an area of the depressor blade not intended to enter the patient's mouth. This is supported by the '019 patent's disclosure of tongue contact portion 20, which is said to be able to bear a flavor coating 22, but not any type of printed image element.

U.S. Pat. No. 3,867,927, entitled ‘Tongue Blade Sucker,” issued to Hergott on Feb. 25, 1975, is an example of how the prior art has gone to great lengths to avoid placing any type of decorative image on the portion of the depressor blade intended to enter the patient's mouth. In the '927 patent, while the depressor blade may contain linear graduations 19 and/or a message 20, for any portions thereof located at the end of the blade intended to enter the patient's mouth a strip 17 is used to cover graduations 19 and/or message 20 so as to “provide a surface with which the examiner depresses the patient's tongue . . . ” (See, column 2, lines 53-54)

Another patent bearing writing and/or images on the portion of the depressor blade not intended to enter the patient's mouth is U.S. Pat. No. 5,897,492, entitled “Candy Tongue Depressor,” issued to Feller et al. on Apr. 27, 1999. In the '492 patent it is the distal end 13 of the depressor which contains any such written message and/or image, or the image is located under candy 12 so as to be covered when the depressor is in use in the doctor's office. (See, column 3, lines 30-48).

A key disadvantage of these prior art inventions is that none offers an opportunity for empowerment of the young patient as well as, interactivity between patient and doctor insofar as allowing the patient to select the tongue depressor from among many different ones for use in the oral examination. Interactivity enhances the doctor-patient relationship and diminishes the patient's level of stress and anxiety in relation to the impending oral exam.

A further disadvantage of the prior art inventions is that none are intended to remain complete more than briefly after use in the oral examination. Consequently, this can create a disconnect in the patient's mental association between the item used during the examination and the item the doctor has provided as a “reward” and therefore does little to reduce fear of tongue depressors and oral examinations in future visits to the doctor.

Another disadvantage is that many of the prior art inventions cannot be used with a standard instrument used by doctors that holds a standard shaped tongue depressor and illuminates the patient's mouth. This commonly used instrument, called a self-illuminating tongue depressor handle attachment, accommodates only tongue depressors of standard shape.

A final disadvantage is that many doctors, are likely to discourage consumption of candy by their young patients due to the lack of nutrients in such “empty calorie” products as well as concerns about childhood obesity. The use of tongue depressors that incorporate candy, whether bearing a candy coating or having a candy attachment, might send the wrong message to both patients and their parents that their doctor condones or approves of adding additional candy to their patients' diets.

The present invention makes oral examinations less emotionally traumatic and more enjoyable for young patients. Colorful, visually arresting images imprinted onto the tongue depressor provide a pleasant surprise to a fearful child. Given the opportunity to select from a variety of different images, the young patient is provided with a sense of empowerment, direct involvement and control over this otherwise uncomfortable part of the examination process. The invention creates relationship-enhancing doctor-patient interactivity, encourages patients' cooperation during the exam, and encourages them to be less fearful of, and upset by, tongue depressors, oral exams, and, in general, visits to the doctor.

Upon completion of the examination, the doctor may offer the patient the used tongue depressor, or even a new, unused tongue depressor, as an entertaining keepsake or “reward” for the patient's cooperation, thereby prolonging the enjoyed experience so that the child will retain positive associations with tongue depressors, oral examinations and doctors in the future. Furthernore, by making tongue depressors available to young patients for use as playthings outside of the medical environment, children may more easily overcome their fears and discomfort concerning tongue depressors, oral exams and doctors.

It is thus an object of the present invention to provide a tongue depressor that diminishes patients' fear of oral exams generally, and tongue depressors specifically, on an ongoing basis, thereby making the examination a more pleasant experience for patients, parents and doctors. As the patient learns to enjoy tongue depressors rather than fear them, the patient's positive association with oral exams and tongue depressors will carry over into future examinations, making them more cooperative and speeding the oral examination process.

In addition, the present invention's wide variety of appealing images inspires the patient to look forward to future examinations, rather than dread them, because of the opportunity for another gratifying choice among the tongue depressor images.

The present invention may be given to the patient as a take-home “reward” or keepsake after completion of the oral exam. Since there is no change to the device before, during or after the examination, the patient can familiarize him/herself with and enjoy tongue depressors outside of the medical environment indefinitely. This helps to break down the youthful patient's perception of fear towards the tongue depressor as a medical instrument and also reduces anxiety for future oral exams.

The present invention may also have the effect of saving the doctor's time in conducting examinations. Upon successfully obtaining the cooperation of a young patient, the oral exam may proceed more quickly.

The present invention is also designed to be capable for use with a self-illuminating tongue depressor handle attachment. The handle attachment accommodates only tongue depressors of standard size and shape. The standard size for use on youthful patients (children) is the standard junior size, which is 5½″ long by ⅝″ wide by 1/16″ thick. The standard shape resembles an ordinary popsicle stick. However, the standard junior size is substantially longer, wider and thinner than an ordinary popsicle stick, which is 4½″ long by ⅜″ wide, with a thickness of 1/12″.

It is the opinion of at least one doctor, that a popsicle stick (1) is too narrow to depress a child's tongue sufficiently to provide a clear view of the tonsils, and (2) would not have the flexibility to comfortably push down on the end that is not inside the patient's mouth and (3) would be too short to allow the doctor to hold it at a comfortable distance from the patient's mouth. However, popsicle stick sized tongue depressors are used on infant patients.

An oral examination of a young patient can also be performed using a tongue depressor of an adult standard size, which is 6″ long by ¾″ wide by 1/16″ thick or a senior standard size which is 6″ long by 11/16″ wide by 1/16″ thick.

### SUMMARY OF THE INVENTION

In accordance with the invention, a method and apparatus for motivating cooperation amongst youthful patients during physical examinations, and specifically oral examinations, is provided. The apparatus, which is an innovative tongue depressor, comprises a blade having first and second sides and first and second tongue contact portions located along the first and second sides, respectfully. An image appears along at least a portion of at least the first tongue contact portion of the first side of the tongue depressor, wherein any portion of the tongue depressor blade may be received within the mouth of the patient, even if the portion of the blade of the tongue depressor has the image appearing therealong. Another image, or possibly an identical image may appear on at least the second tongue contact portion of the second side of the blade of the depressor. Here again, whether this portion of blade is meant to go into the patient's mouth or not, the image may appear therealong. The image is made from ink approved by the U.S. Food and Drug Administration for application to items to be received within a person's mouth.

Accordingly, it is an object of the invention to provide an improved tongue depressor apparatus. Still another object of the invention is to provide an improved tongue depressor apparatus that has an image appearing on at least one side of the blade of the depressor.

Yet another object of the invention is to provide an improved tongue depressor apparatus having an image appearing on both sides of the blade of the depressor. A further object of the invention is to provide an improved tongue depressor apparatus having different images on the two different sides of the blade of the depressor, or multiple images along one or both sides of the blade of the depressor.

Yet a further object of the invention is to provide an improved tongue depressor apparatus wherein the image is made from FDA approved inks for application to items to be received within a person's mouth.

Still further objects of the invention are to:
(1) empower young patients by permitting them to select the particular tongue depressor to be used by the doctor, prior to the oral examination.
(2) give young patients a sense of control and ownership within an event that is otherwise entirely beyond their control.
(3) add an element of fun, interactivity and relationship building to the oral examination process.
(4) allow the practitioner to immediately reward, and thus reinforce, the child for cooperative behavior.
(5) break the unhealthy tradition, which is increasingly being disavowed by medical practitioners, of rewarding children with candy or allowing uncontrolled consumption of sweets.

The invention accordingly comprises assemblies possessing the features, properties and the relation of components which will be exemplified in the products hereinafter described, and the scope of the invention will be indicated in the claims.

### BRIEF DESCRIPTION OF THE DRAWINGS

For a fuller understanding of the invention, reference is made to the following description, taken in connection with the accompanying drawings, in which:

FIG. 1 is a top planar view of the tongue depressor; and

FIG. 2 is a perspective view of the same tongue depressor.

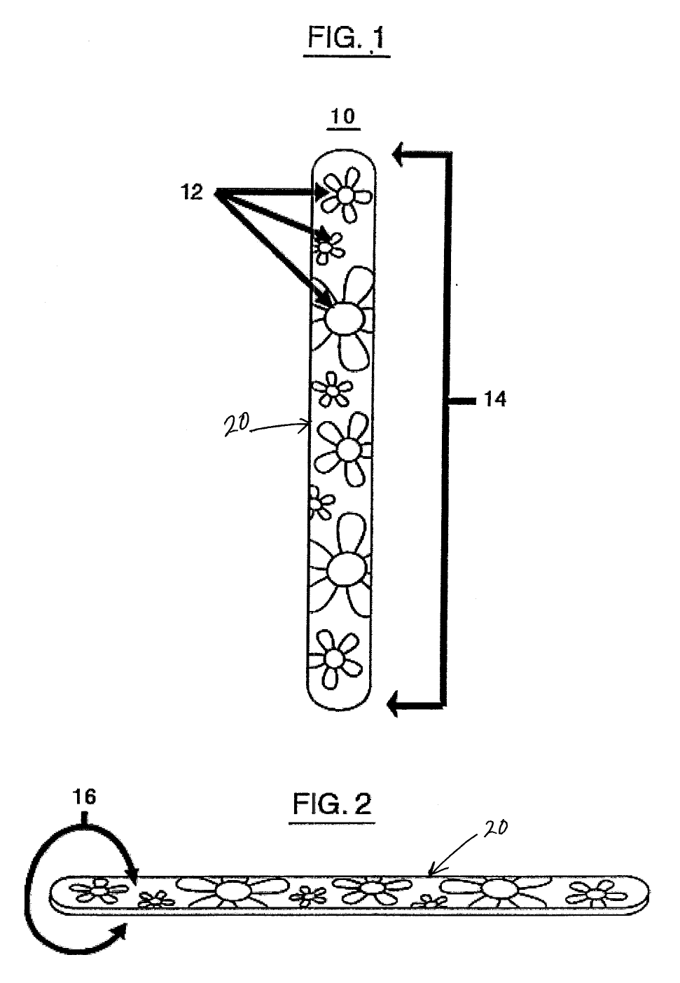

### DETAILED DESCRIPTION OF THE PREFERRED EMBODIMENTS

Referring to the figures, tongue depressor 10 is comprised of a thin, flat, non-edible stick with a longitudinal axis made of a nontoxic material such as wood, wood composite, plastic, plastic composite, cardboard or cardboard composite, or any other applicable material known in the trade of making tongue depressors. Tongue depressor 10 can be a standard size or any other size whose length and width is appropriate for use as a tongue depressor. In addition, tongue depressor 10 may be sterile or non-sterile, and may contain a flavor coating, or could simply be flavorless.

Tongue depressor 10 bears vivid images 12 printed directly onto the blade 20 by way of screen printing or other known printing methods. In the shown preferred embodiment, images 12 are of flowers, but the invention anticipates that images 12 can include any known image or writing, including, but not limited to, patterns, images, cartoon characters, corporate logos, advertising messages, or any other image that is appealing to children.

Tongue depressor 10 is printed using non-toxic ink 14 that has been approved by the U.S. Food and Drug Administration or other approval process to be safely used inside the mouth, on the tongue.

It is also to be understood from the figures that tongue depressor 10 may be printed with image 12 on one of its sides 16 or both of its sides 16, as is best seen in FIG. 2. In addition, the invention anticipates that image 12 may consist of the same or different image(s) on either of sides 16 and/or along either of sides 16; i.e., multiple images may appear on either of sides 16, and/or on both. Further, image 12 can be printed along the entire surface of blade 20, or along only a portion or portions thereof. Images 12 can also be printed in one color or multiple colors.

By way of a non-limiting example, the method of this invention and the use of the improved tongue depressor will be illustrated through a possible patient examination procedure. It will be readily apparent to one skilled in the art, however, that this invention is equally appropriate for use in examinations conducted in other ways.

As a first step, prior to oral examination, a doctor gives a young patient one or more of the improved tongue depressors to look at, hold, play with and/or talk about.

As a second step, the doctor asks the patient to choose the improved tongue depressor with his or her favorite image to be used for the oral exam.

As a third step, the doctor uses the information gleaned from the young patient's selection to initiate a discussion with the patient. For example, if the image depicts a basketball, the doctor may ask, “do you play basketball?” or if the image depicts a red fire engine, the doctor may ask, “is red your favorite color?”

As a fourth step, the doctor directs the discussion to the oral examination procedure and the rewards enjoyed by cooperative patients. The doctor may offer the improved tongue depressor as a reward to the patient for cooperating in the oral examination.

As a fifth step, the doctor asks the patient to open his or her mouth and uses the improved tongue depressor to conduct the oral examination.

As a sixth step, the doctor eases patient tension by talking about the image as he or she conducts the examination (e.g., “open your mouth and let the fire engine see your throat!”).

As a seventh step, the doctor may give the improved tongue depressor to the patient.

It will thus be seen that the objects set forth above, among those made apparent from the preceding description, are efficiently attained, and since certain changes may be made in the above constructions without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of the scope of the invention, which, as a matter of language, might be said to fall therebetween.

What is claimed is:

1. An improved tongue depressor for receipt within a mouth of a patient during an examination, comprising:
a blade comprising first and second sides separated by a thickness of said blade, said blade further comprising first and second tongue contact portions located along said first and second sides, respectively with said blade having a rounded profile on a first and second end; and
an image appearing along at least a portion of at least said first tongue contact portion of said first side; wherein said improved tongue depressor is more than 4½″ long and more than ⅜″ wide, said tongue depressor is not coated with flavor, and any portion of said tongue depressor blade may be received within said mouth of said patient during said examination, even if said any portion of said tongue depressor has said image appearing therealong.

2. An improved tongue depressor as recited in claim 1, said image appearing along both said first tongue contact portion and said second tongue contact portion of said blade.

3. An improved tongue depressor as recited in claim 1, further comprising another image appearing along at least a portion of at least said second tongue contact portion of said blade, wherein even a portion of said improved tongue depressor blade bearing said another image may be received into said mouth of said patient.

4. An improved tongue depressor as recited in claim 3, wherein said image and said another image are the same.

5. An improved tongue depressor as recited in claim 3, wherein said image and said another image are different.

6. An improved tongue depressor as recited in claim 3, wherein said image and said another image are made of non-toxic ink for application to items to be received within a person's mouth.

7. An improved tongue depressor as recited in claim 1, wherein said improved tongue depressor is made of wood.

8. An improved tongue depressor as recited in claim 1, wherein said improved tongue depressor is made of plastic.

9. An improved tongue depressor as recited in claim 1, wherein said improved tongue depressor is made of cardboard.

10. An improved tongue depressor as recited in claim 1, wherein said improved tongue depressor is sterile.

11. An improved tongue depressor for motivating the cooperation of youthful patients in oral examinations, comprising:
a tongue depressor that is more than 4½″ long and more than ⅜″ wide, and further comprises a blade having two elongated, substantially flat surfaces for restraining said tongue of said patient, at least one of said two surfaces bearing an image along at least a portion thereof intended to be inserted into said patient's mouth, with said blade having a rounded profile on a first and second end, said image being made from non-toxic ink, and any portion of said tongue depressor blade may be received within said mouth of said patient during said examination, even if said any portion of said tongue depressor has said image appearing therealong.

12. An improved tongue depressor as recited in claim 1, wherein said image substantially covers the entire surface of said blade.

13. An improved tongue depressor as recited in claim 1, wherein said image include images that appeal to children.

14. An improved tongue depressor as recited in claim 1, wherein said image consists of at least two colors.

15. An improved tongue depressor as recited in claim 3, wherein said image and said another image contain images with repeating patterns from end to end of said blade.

16. A method of conducting an oral examination, comprising:
a first step, wherein prior to the oral examination, a doctor gives a patient one or more of an improved tongue depressor having an image thereon;
a second step, wherein the doctor asks the patient to select an image from among the said images on the improved tongue depressors;
a third step, wherein the doctor uses the information gleaned from the patient's selected image to initiate a discussion with the patient;
a fourth step, wherein the doctor discusses the oral examination and further discusses a reward that is provided to patients who are cooperative;
a fifth step, wherein the doctor asks the patient to open his or her mouth and uses the improved tongue depressor having thereon the selected image to conduct the oral examination; and
a sixth step, wherein the doctor talks about the selected image as he or she conducts the oral examination;
a seventh step, wherein the doctor gives the improved tongue depressor to the patient;
wherein said improved tongue depressor is more than 4½″ long and more than ⅜″ wide, the blade having two elongated, substantially flat surfaces, with said blade having a rounded profile on a first and second end, at least one of said two surfaces bearing an image along substantially the entire surface thereof, said image being made from non-toxic ink, and said blade is not coated with flavor, and both ends of said blade may be received within the mouth of said patient.

17. The method as recited in claim 16, wherein said image includes child-oriented images that appeal to children.

18. The method as recited in claim 16, wherein said image and said another image contain images with repeating patterns from end to end of said blade.
